(19) United States
(12) Patent Application Publication
(10) Pub. No.: US 2026/0243658 A1
(43) Pub. Date: Aug. 20, 2026

(54) CONTAINER HOLDER DEVICE

(71) Applicant: GASPOROX, Lund (SE)

(72) Inventors: Rikard WELLANDER, Annelöv (SE); Jonathan AHLSE, Malmö (SE)

(21) Appl. No.: 18/869,827

(22) PCT Filed: May 31, 2023
(86) PCT No.: PCT/EP2023/064626

§ 371 Date: Nov. 26, 2024

(30) Foreign Application Priority Data
Jun. 1, 2022 (SE) 2250658-8

(51) Int. Cl. G01N 21/01; G01N 33/00

(52) U.S. Cl. CPC G01N 21/01; G01N 33/0027

(57) ABSTRACT

A container holder (1) for use during non-intrusive optical detection of gases inside a container (2) comprises a first part (3) and a second part (4). The first part (3) has a container contact surface and the second part (4) comprises a container contact surface (5). The contact surfaces of the first and second parts face one another when the two parts (3, 4) are assembled. A light aperture is arranged on the contact surface of the first part (3) and a light receiving element (6) is arranged at the contact surface (5) of the second part (4). The container contact surface (5) of the second part (4) is arranged on a detachable element (7) and the detachable element (7) comprises an abutment flange (8) extending at an angle from the contact surface at the upper end of the contact surface (5). The abutment flange (8) is arranged to be in contact with the bottom of a container (2). The detachable element (7) further comprises a spring biased lifting element (9) arranged at the lower end of the contact surface (5) and arranged to hold a container (2) pressed up-side down against the abutment flange (8).

### TECHNICAL FIELD

The present invention relates to a container holder for use during non-intrusive optical detection of gases inside a container. The holder comprises a first part and a second part, wherein the first part has a container contact surface and the second part comprises a container contact surface. The contact surfaces of the first and second parts face one another when the two parts are assembled. A light aperture is arranged on the contact surface of the first part and a light receiving element is arranged at the contact surface of the second part.

The present invention also relates to a method of detecting gases inside a container in a non-intrusive way.

### BACKGROUND

Many parenteral drugs are filled in glass containers like vials and ampoules. Oxygen sensitive drugs need to be produced and filled with a low residual oxygen in the headspace of the container. Often a nitrogen overlay is used during the aseptic filling process. Multiple vacuum/nitrogen cycles are used for even lower residual oxygen levels. The GMP (Good Manufacturing Practice) regulations request a verification of the effectiveness of the process.

Instruments are available that enable accurate and fast non-destructive measurement of residual oxygen in the headspace of the vial or ampoule for headspace analysis or leak testing, CCIT-Container Closure Integrity Testing. For instance, a container with a sample is placed in the holder. A light beam is sent in the headspace of the vial, probing the gas inside and providing instant result.

The containers of interest to measure can be e.g. pharmaceutical vials or ampoules. These can be in a large variety of different sizes and shapes. To have good holders for correct positioning is challenging given the different available sizes and shapes. A flexible format that can be adjusted to hold different formats is thus beneficial.

Another alternative is to place a sample into a closely fitting chamber that is specially designed based on sample type and size of vial or ampoule. Once enclosed, vacuum is applied to the chamber. The vacuum level inside the chamber and any changes in vacuum are measured over a predetermined test time using absolute and differential pressure transducers. An increase in pressure that exceeds the predetermined pass or fail parameters indicates the presence of a leak.

### SUMMARY

It is an object of the present invention to provide an improved or at least an alternative container holder.

Critical aspects when performing measurements on containers based on light beams sent through the container is to avoid getting the ambient air outside the container to interfere with the measurement. The positioning of the container and avoiding getting ambient air offsets, or varying ambient offset is key, meaning measuring in the well-defined cylindrical part of the container and having the light beam pass a well-defined path is important. Also, that the light beam passes the gas filled part of the container is necessary for correct measurement. As many containers, vials and ampoules have a high fill level, it is challenging to have a section large enough of the cylindrical part free of gas. A way to solve this is to turn the container upside down which is one of the aspects of the present disclosure.

According to a first aspect of the present disclosure a container holder for use during non-intrusive optical detection of gases inside a container is provided. The holder comprises a first part and a second part, wherein the first part has a container contact surface and the second part comprises a container contact surface. The contact surfaces of the first and second parts face one another when the two parts are assembled. A light aperture is arranged on the contact surface of the first part and a light receiving element is arranged at the contact surface of the second part. The container contact surface of the second part is arranged on a detachable element. The detachable element comprises an abutment flange extending at an angle from the contact surface at the upper end of the contact surface. The abutment flange is arranged to be in contact with the bottom of a container. The detachable element further comprises a spring biased lifting element arranged at the lower end of the contact surface and arranged to hold a container pressed up-side down against the abutment flange. The light receiving element may be arranged adjacent the upper end of the contact surface close to the abutment flange.

Vials and especially ampoules can be difficult to measure since the level to which they are filled varies. An ampoule that is filled with some content might be filled to a level above the cylindrical part of the ampoule. When sending a light through an empty part of the ampoule that is not cylindrical, for instance when the container diameter varies, the light is deflected in an un-predictable manner. Thus, by arranging the container, i.e., most of the times a vial or an ampoule, upside down, it is more likely that an empty part of the container will be a cylindrical part of the container such that the light will travel through the container in a predictable manner. Also, the spring biased lifting element ensures the bottom of a container is always pushed against the abutment flange.

According to a further aspect of the present disclosure, the detachable element is spring biased in the second part to push a container towards the contact surface of the first part such that the container is held between the contact surfaces of the two parts when in use.

By having at least one contact surface spring biased, the container is more securely held in position during measurement.

According to another aspect of the present disclosure, the contact surface of the second part has a groove extending at least a part of the distance between the upper and lower ends of the contact surface.

The groove facilitates getting the container in a desired position such that the container may get aligned with the groove.

According to yet another aspect of the present disclosure, the groove has a V-shaped cross section. Also, preferably the groove is of a size that relative to the size of the container, only one position is possible. For instance, for a cylindrical container, the V-shaped groove is of a size that it provides two contact surfaces or contact lines along the cylindrical container.

According to one aspect of the present disclosure, the light receiving element is arranged in the groove. In other words, the light receiving element is preferably arranged close to the abutment flange.

According to yet one aspect of the present disclosure, the light receiving element is arranged with a spring such that it pushes against the surface of a container when in use. Thus, even with variations in the sizes of the containers. The light receiving element is always in direct contact with the container such that the light does not travel through ambient air surrounding the container. Further, in this context, a spring comprises a standard spring or for instance a poron cushion.

Alternatively, the light receiving element comprises a detector surface arranged in the light receiving element such that the detector surface is at a constant distance from the vial or ampoule when the light receiving element is pushed against the vial or ampoule. Thus, also the distance traveled by light in ambient air will be constant. The light receiving element is in this example a holder for a detector surface.

According to an alternative aspect of the present disclosure, the lifting element comprises two parallel rod elements, such that the rod elements is arranged to grip the neck of an ampoule. An alternative to two rods could be a plate with an elongate slit in it. This could possibly be considered as two “flat” rods.

An advantage of this is that an ampoule is that it will facilitate the placing of an ampoule in the holder since it may be clearer to a user on how to place it.

According to another alternative aspect of the present disclosure, the container contact surface of the second part is arranged in a holding part and the holding part is movably arranged in a parallelogram in the detachable element between a position at a first distance from the contact surface of the first part and a second distance from the contact surface of the first part.

A benefit of this arrangement is that the second part with the detachable element can be used for various container formats.

According to a preferred aspect of the holding part being movably arranged in the detachable element, a line between said positions of the holding part is not perpendicular to the contact surfaces of the first and second parts. For instance, if the light is sent through the container at an angle that is not perpendicular to the axis of the container, changing the type a container as regards the diameter allows for the light to always be received by the light receiving element.

Preferably, said an angle between said line and a line perpendicular to the contact surfaces of the first and second parts is between 1 and 45 degrees.

According to yet a further aspect of the present disclosure, the detachable element is hinged to the second part of the holder. Having the detachable element hinged to the second part facilitates the handling as regards inserting and removing the sample containers. The detachable element being hinged to the second part is made such that the first and second parts of the container holder can stay assembled and the detachable element is always inserted and detached in a correct manner.

According to an aspect of the present disclosure, the abutment flange is movably arranged relative the contact surface of the second part. This allows for using the same container holder for a larger size variety in containers.

According to yet an aspect of the present disclosure, the first part and the second part are magnetically coupled to one another. Should the second part need to be replaced for any reason, the magnetic coupling facilitates this replacement. Preferably, the contact portion between the first and second parts of the holder have male/female coupling such that the two parts are always in a correct position relative one another.

The light aperture arranged on the contact surface of the first part comprises a collimating lens adapted for laser light according to a further aspect of the present disclosure.

The light receiving element is according to one aspect of the present disclosure one of a charge-coupled device, a position-sensitive detector, photoelectric sensor, and a complementary metal oxide semiconductor.

Yet another aspect of the present disclosure provides a method of non-intrusive optical detection of gases inside a container. The method comprises placing a container such as a vial or ampoule upside down in a holder, sending a light from one side of and through the vial or ampoule adjacent the bottom of the vial or ampoule turned upside down, and detecting the light on the opposite side of the vial or ampoule. This method is preferably carried out using a container holder according to the present disclosure.

Further features of, and advantages with, the present invention will become apparent when studying the appended claims and the following description. The skilled person realize that different features of the present invention may be combined to create embodiments other than those described in the following, without departing from the scope of the present invention as defined by the claims.

### BRIEF DESCRIPTION OF THE DRAWINGS

The above, as well as additional objects, features and advantages of the present invention, will be better understood through the following illustrative and non-limiting detailed description of exemplary embodiments of the present invention, wherein:

FIG. 1a is a side view of a part of a holder part according to the present disclosure,

FIG. 1b is a front view of a lifting element according to the present disclosure,

FIGS. 2 and 3 side views of a lifting element according to the present disclosure,

FIG. 4 is a side view of a detachable part according to the present disclosure,

FIG. 5 is schematic top view of a holder part according to the present disclosure,

FIG. 6 a partial side view of the container holder according to the present disclosure,

FIG. 7 is a perspective view of a measuring unit with a container holder according to the present disclosure,

FIG. 8 is a side view of the measuring unit of FIG. 7,

FIG. 9 is a perspective view of a measuring unit with a container holder according to the present disclosure, and

FIG. 10 is a sideview of the measuring unit of FIG. 9.

### DETAILED DESCRIPTION OF EXAMPLE EMBODIMENTS OF THE INVENTION

The present invention will now be described more fully hereinafter with reference to the accompanying drawings, in which exemplary embodiments of the invention are shown. The invention may, however, be embodied in many different forms and should not be construed as limited to the embodiments set forth herein; rather, these embodiments are provided for thoroughness and completeness. Like reference character refer to like elements throughout the description.

With reference to the figures, a container holder 1 for use during non-intrusive optical detection of gases inside a container 2 is shown. The holder 1 comprises a first part 3 and a second part 4, see FIGS. 8 and 10. The first part 3 has a container contact surface and the second part 4 comprises a container contact surface 5, wherein the contact surfaces of the first and second parts face one another when the two parts 3, 4 are assembled. A light aperture (not shown) is arranged on the contact surface of the first part 3 and a light receiving element 6 is arranged at the contact surface 5 of the second part 4.

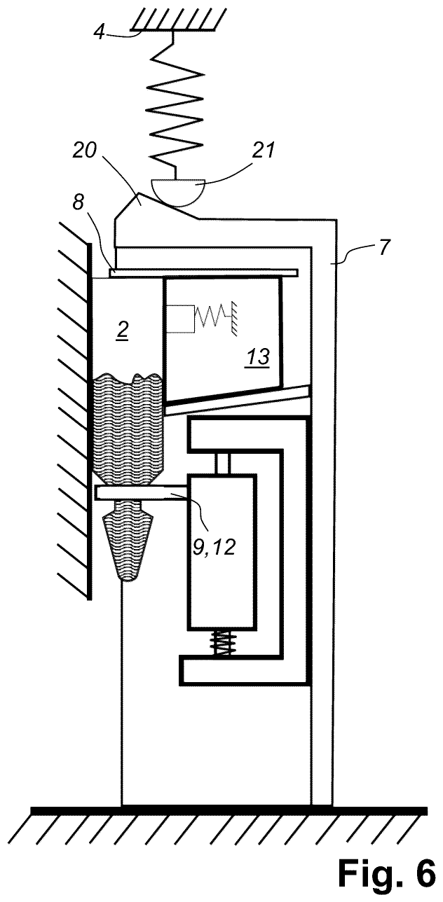

The container contact surface 5 of the second part 4 is arranged on a detachable element 7. The detachable element 7 comprises an abutment flange 8 extending at an angle from the contact surface at the upper end of the contact surface 5. In the shown embodiment the abutment flange 8 is arranged perpendicular to the contact surfaces of the two parts 3, 4. The abutment flange 8 is arranged such that it is in contact with the bottom of a container 2, see for instance FIG. 6. The detachable element 7 further comprises a spring biased lifting element 9 arranged at the lower end of the contact surface 5 and arranged to hold the container 2 pressed up-side down against the abutment flange 8.

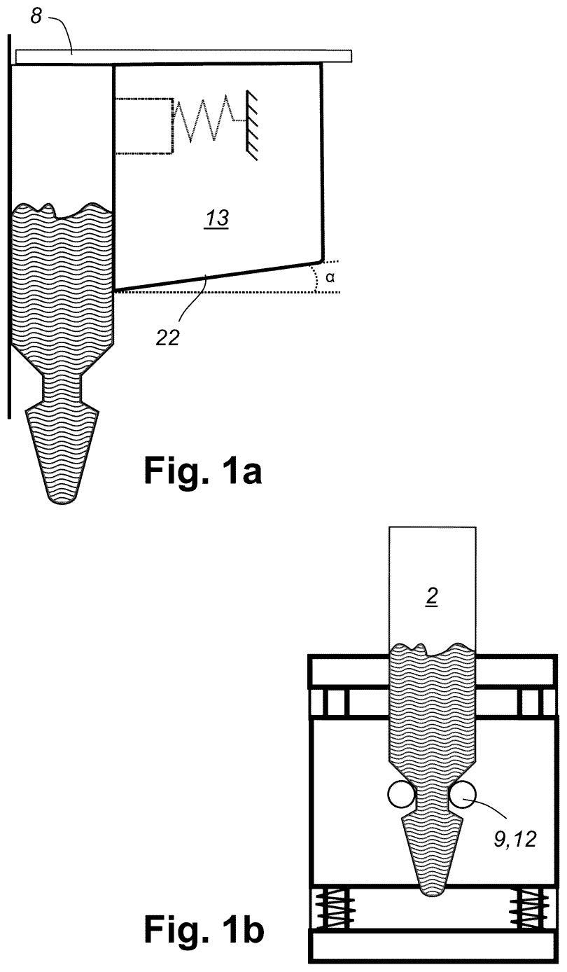

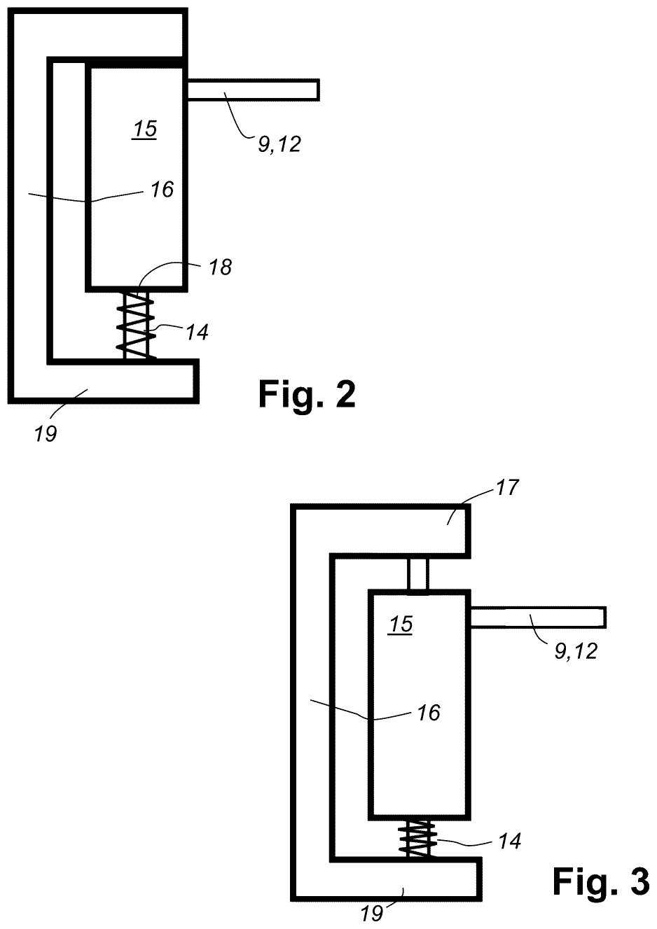

FIGS. 1b, 2 and 3 show this lifting element 9. The lifting element comprises in the shown embodiment two parallel rod elements 12, such that the rod elements 12 grips the neck of an ampoule 2, see for instance FIG. 1b. In FIG. 2, the lifting element 9 is in its highest position as compared to what is shown in FIG. 3 where the springs 14 are somewhat compressed. The lifting element 9 is arranged with a block element 15 holding the rod elements 12. The block element 15 is arranged inside a frame 16 that limits the movement of the block element 15, the frame 16 having two upper shoulders 17. The block element 15 is movably arranged on two rods 18 that are fixed to the frame 16 between the upper shoulders 17 and lower shoulders 19.

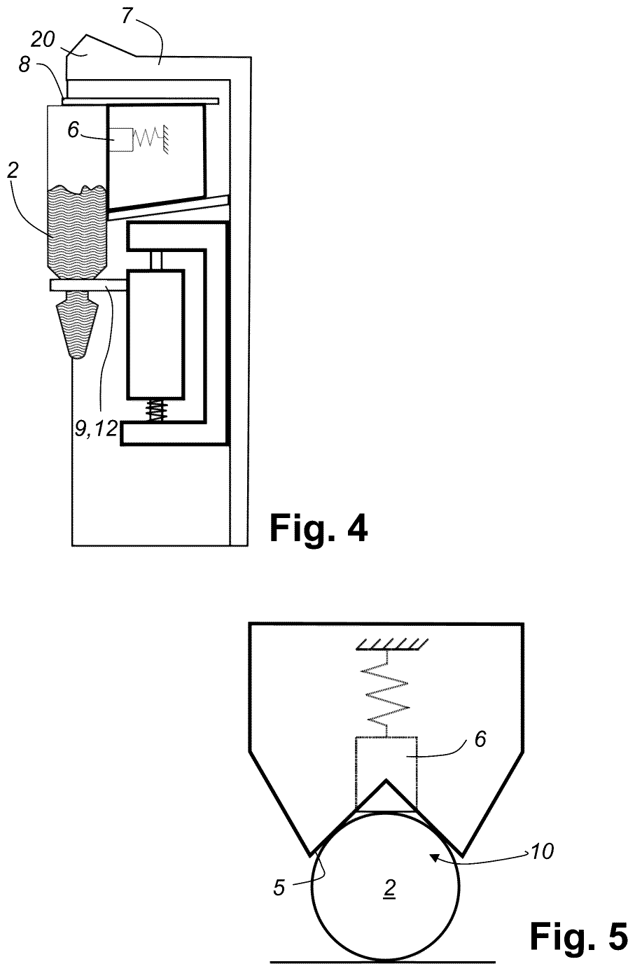

The detachable element 7 is spring biased in the second part 4 to push a container 2 towards the contact surface of the first part 3 such that the container 2 is held between the contact surfaces of the two parts when in use. This is in the shown embodiment achieved by a combination of a ridge 20, see FIGS. 4 and 6, and a spring biased pushing element 21, see FIG. 6. The pushing element 21 pushes on one side of the ridge 20 such as to press the detachable element 7 into a position where the detachable element 7 and the second part 4 fully assembled.

The contact surface 5 of the second part 4 has a groove 10 extending at least a part of the distance between the upper and lower ends of the contact surface 5, see FIG. 5. In FIG. 5, the groove 10 is V-shaped.

As further can be seen in FIG. 5, a light receiving element 6 is arranged in the groove 10. Going back to FIG. 4, the light receiving element 6 may be arranged adjacent the upper end of the contact surface 5 close to the abutment flange 8. The light receiving element 6 is arranged with a spring such that it pushes against the surface of a container 2 when in use. In this manner, the light receiving element can be in direct contact with a vial or an ampoule 2. The abutment flange 8 is movably arranged relative the contact surface 5 of the second part 4 holding the abutment flange 8 to the holding part 13.

The container contact surface 5 of the second part 4 is arranged in a holding part 13, wherein the holding part 13 being movably arranged in a parallelogram in the detachable element 7 between a position at a first distance from the contact surface of the first part and a second distance from the contact surface of the first part. A line 22 between said positions of the holding part is not perpendicular to the contact surfaces of the first and second parts 3, 4. An angle α between said line 22 and a line perpendicular to the contact surfaces of the first and second parts 3, 4 is between 1 and 45 degrees, see FIG. 1.

The detachable element 7 is preferably hinged to the second part 4 of the container holder 1. When the detachable element 7 is detached from the second part 4 it is possible to insert or remove a vial or ampoule 2.

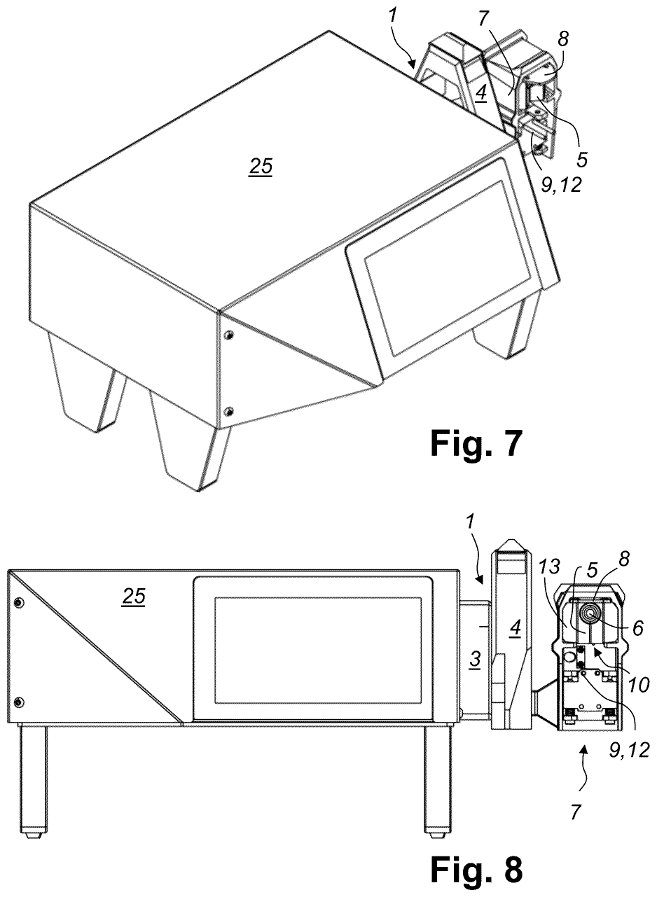

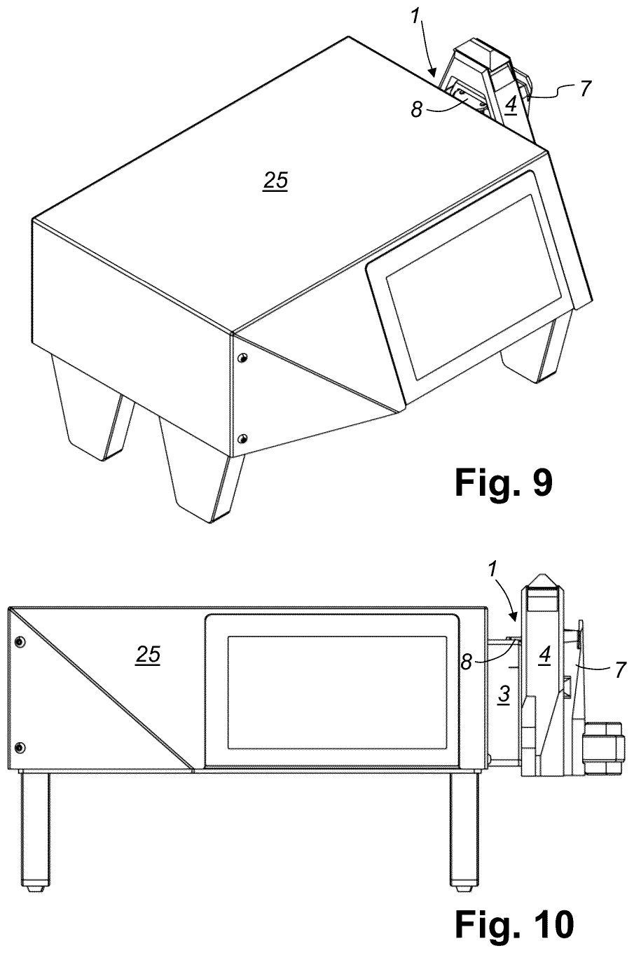

In FIGS. 7 to 10 an entire measuring unit 25 is shown with the first and second parts 3, 4. FIGS. 7 and 8 show the unit 25 when the detachable element 7 is open, i.e., when a container can be inserted or removed whereas FIGS. 9 and 10 show the unit 25 when the detachable element 7 is assembled in the second part 4.

As used herein, the singular forms “a”, “an” and “the” are intended to include the plural forms as well, unless expressly stated otherwise. It will be further understood that the terms “includes,” “comprises,” “including” and/or “comprising,” when used in this specification, specify the presence of stated features, integers, steps, operations, elements, and/or components, but do not preclude the presence or addition of one or more other features, integers, steps, operations, elements, components, and/or groups thereof. It will be understood that when an element is referred to as being “connected” or “coupled” to another element, it can be directly connected or coupled to the other element or intervening elements may be present. Furthermore, “connected” or “coupled” as used herein may include wirelessly connected or coupled. As used herein, the term “and/or” includes any and all combinations of one or more of the associated listed items.

While the embodiment of the present invention as described and illustrated herein, those of ordinary skill in the art will readily envision a variety of other means and/or structures for performing the functions and/or obtaining the results and/or one or more of the advantages described herein, and each of such variations and/or modifications that are deemed to be within the scope of the present invention as defined by the claims.

1. A container holder for use during non-intrusive optical detection of gases inside a container the holder comprising a first part and a second part, wherein the first part has a container contact surface and the second part comprises a container contact surface wherein the contact surfaces of the first and second parts face one another when the two parts are assembled, wherein a light aperture is arranged on the contact surface of the first part and a light receiving element is arranged at the contact surface of the second part, wherein:
the container contact surface of the second part is arranged on a detachable element;
the detachable element comprises an abutment flange extending at an angle from the contact surface of the second part at the an upper end of the contact surface of the second part;
the abutment flange is configured to be in contact with the a bottom of the container;
the detachable element further comprises a spring biased lifting element arranged at the a lower end of the contact surface of the second part and arranged to hold the container pressed up-side down against the abutment flange.

2. The container holder according to claim 1, wherein the detachable element is spring biased in the second part to push the container towards the contact surface of the first part such that the container is held between the contact surfaces of the two parts when in use.

3. The container holder according to claim 1, wherein the contact surface of the second part has a groove extending at least a part of the a distance between the upper and lower ends of the contact surface of the second part.

4. The container holder according to claim 3, wherein the groove has a V-shaped cross section.

5. The container holder according to claim 3, wherein the light receiving element is arranged in the groove of the contact surface of the second part.

6. The container holder according to claim 5, wherein a spring is configured to push the light receiving element against the a surface of a the container when in use.

7. The container holder according to claim 1, wherein the lifting element comprises two parallel rod elements such that the rod elements are arranged to grip the a neck of an ampoule.

8. The container holder according to claim 1, wherein the container contact surface of the second part is arranged in a holding part the holding part being movably arranged in a parallelogram in the detachable element between a first position at a first distance from the contact surface of the first part and a second position at a second distance from the contact surface of the first part.

9. The container holder according to claim 8, wherein a line between the first position and the second position of the holding part is not perpendicular to the contact surfaces of the first and second parts

10. The container holder according to claim 9, whereinan angle between the line and a line perpendicular to the contact surfaces of the first and second parts is between 1 and 45 degrees.

11. The container holder according to claim 8, wherein the detachable element is hinged to the second part of the container holder.

12. The container holder according to claim 1, wherein the abutment flange is movably arranged relative the contact surface of the second part.

13. The container holder according to claim 1, wherein the first part and the second part are magnetically coupled to one another.

14. The container holder according to claim 1, wherein the light aperture arranged on the contact surface of the first part comprises a collimating lens adapted for laser light.

15. A method of non-intrusive optical detection of gases inside a container the method comprising,
placing a the container such as a vial or ampoule upside down in a holder,
sending a light from one side of and through the vial or the ampoule adjacent a bottom of the vial or the ampoule turned upside down, and
detecting the light on the an opposite side of the vial or the ampoule.
